(19) United States
(12) Patent Application Publication
(10) Pub. No.: US 2026/0185915 A1
(43) Pub. Date: Jul. 2, 2026

(54) INSPECTION SYSTEM, SUBSTRATE PROCESSING APPARATUS INCLUDING SAME, AND INSPECTION METHOD USING SAME

(71) Applicant: Samsung Electronics Co., Ltd., Suwon-si (KR)

(72) Inventors: Byungchul CHOI, Suwon-si (KR); Taejung KIM, Suwon-si (KR); Byoungchel JEON, Suwon-si (KR); Sanghwan AN, Suwon-si (KR); Soo Yong OH, Suwon-si (KR); Donghyun JANG, Suwon-si (KR); Sang Bae JUNG, Suwon-si (KR)

(21) Appl. No.: 19/242,651

(22) Filed: Jun. 18, 2025

(30) Foreign Application Priority Data
Jan. 2, 2025 (KR) 10-2025-0000290

(51) Int. Cl. G01N 15/06; G01N 33/00; H01L 21/67

(52) U.S. Cl. CPC G01N 15/06; G01N 33/0027; H10P 72/0402

(57) ABSTRACT

An inspection system includes: a gas box configured to store gas, an inspection device configured to receive gas supplied from the gas box to the inspection device and configured to inspect the gas received from the gas box, and an inspection line connecting the gas box to the inspection device and configured to allow gas supplied from the gas box to flow through the inspection device. The inspection device includes: a first sensor configured to detect particles present in the gas flowing through the inspection line, and a second sensor configured to detect target gas present in the gas flowing through the inspection line.

### CROSS-REFERENCE TO RELATED APPLICATION

This application is based on and claims priority to Korean Patent Application No. 10-2025-0000290, filed in the Korean Intellectual Property Office on Jan. 2, 2025, the entire contents of which are hereby incorporated by reference.

### BACKGROUND

### Technical Field

Embodiments of the present disclosure relate to an inspection system, a substrate processing apparatus including the same, and an inspection method using the same.

### Description of the Related Art

Various process gases may be used in semiconductor manufacturing facilities for manufacturing semiconductor devices. A gas box for storing the process gas controls the flow of various gases. However, in a case where toxic gases and the like remain in the gas box or are exposed to the outside air, the gas box may be contaminated.

Contamination of the gas box may deteriorate the quality of the semiconductor device. Thus, it is desirable to determine whether or not the gas box is contaminated and manage which components are contaminated.

### SUMMARY

Embodiments of the present disclosure provide an inspection system, a substrate processing apparatus, and an inspection method capable of inspecting whether a gas box is contaminated.

Embodiments of the present disclosure provide an inspection system, a substrate processing apparatus, and an inspection method that improve the contamination detection capability of a gas box.

According to some embodiments of the present disclosure, an inspection system may include a gas box configured to store gas, an inspection device configured to receive gas supplied from the gas box to the inspection device and configured to inspect the gas received from the gas box, and an inspection line connecting the gas box to the inspection device and configured to allow gas supplied from the gas box to flow through the inspection device; the inspection device includes a first sensor configured to detect particles present in the gas flowing through the inspection line, and a second sensor configured to detect target gas present in the gas flowing through the inspection line.

According to some embodiments of the present disclosure, a substrate processing apparatus may include: a chamber for processing a substrate, a gas box configured to store gas to be supplied to the chamber, a foreline connected to the chamber configured to discharge the gas from the chamber, and an inspection device configured to connect at least one of the foreline or the chamber to the gas box, and an inspection line connecting the gas box to the inspection device and configured to allow the gas supplied from the gas box to flow through the inspection device, in which the gas box includes a gas line configured to allow the gas to move therethrough, a gas valve configured to selectively open and close the gas line, a flow controller configured to control a flow rate of the gas moving through the gas line and disposed downstream of the gas valve, and a purge supply configured to supply a purge gas to the gas line to purge the gas line, and disposed upstream of the gas valve and the flow controller, and wherein the inspection device includes a particle sensor configured to detect particles present in the gas flowing through the inspection line, and a gas sensor configured to detect target gas present in the gas flowing through the inspection line.

According to some embodiments of the present disclosure, an inspection method may include connecting an inspection device with a gas box, connecting the inspection device with at least one of a chamber or a foreline connected to the chamber, supplying gas from the gas box to the inspection device, and determining whether the gas box is contaminated, through the inspection device.

According to some embodiments of the present disclosure, the inspection system, substrate processing apparatus, and the inspection method are capable of intuitively determining whether the gas box is contaminated.

According to some embodiments of the present disclosure, the inspection system, substrate processing apparatus, and the inspection method are capable of determining whether outside air is introduced to the system, for example when a sensor is replaced.

According to some embodiments of the present disclosure, the inspection system, substrate processing apparatus, and the inspection method are capable of removing and detecting particles or residual gas.

According to some embodiments of the present disclosure, the inspection system, substrate processing apparatus, and the inspection method are capable of being applied to various substrate processing apparatuses.

### BRIEF DESCRIPTION OF DRAWINGS

FIGS. 1 and 2 illustrate a substrate processing apparatus according to some embodiments of the present disclosure.

FIG. 3 illustrates a configuration of a gas box according to some embodiments of the present disclosure.

FIGS. 4 and 5 illustrate a configuration of an inspection system according to some embodiments of the present disclosure.

FIGS. 6 and 7 illustrate a configuration of an inspection system according to some embodiments of the present disclosure.

FIGS. 8 and 9 illustrate flow charts showing inspection methods according to some embodiments of the present disclosure.

FIG. 10 illustrates a flow chart showing an inspection method according to some embodiments of the present disclosure.

FIG. 11 illustrates a graph showing a result according to an inspection method according to some embodiments of the present disclosure.

FIGS. 12 and 13 illustrate diagrams for explaining an inspection system according to some embodiments of the present disclosure.

FIG. 14 illustrates a flow chart showing an inspection method according to some embodiments of the present disclosure.

FIG. 15 illustrates a diagram for explaining a gas box according to some embodiments of the present disclosure.

FIG. 16 illustrates a flow chart showing an inspection method according to some embodiments of the present disclosure.

### DETAILED DESCRIPTION

An inspection system according to some embodiments of the present disclosure, a substrate processing apparatus including the same, and an inspection method using the same will be described in detail, with reference to the accompanying drawings.

Items described in the singular herein may be provided in plural, as can be seen, for example, in the drawings. Thus, the description of a single item that is provided in plural should be understood to be applicable to the remaining plurality of items unless context indicates otherwise

It will be understood that when an element is referred to as being “connected” to or “on” another element, it can be directly connected to or on the other element or intervening elements may be present.

Ordinal numbers such as “first,” “second,” “third,” etc. may be used simply as labels of certain elements, steps, etc., to distinguish such elements, steps, etc. from one another. Terms that are not described using “first,” “second,” “third,” etc., in the specification, may still be referred to as “first” or “second” or “third” in a claim. In addition, a term that is referenced with a particular ordinal number (e.g., “first” in a particular claim) may be referenced elsewhere without an ordinal number or with a different ordinal number (e.g., “second” in the specification or another claim). Throughout the specification, when a component is described as “including” a particular element or group of elements, it is to be understood that the component is formed of only the element or the group of elements, or the element or group of elements may be combined with additional elements to form the component, unless the context indicates otherwise. The term “consisting of,” on the other hand, indicates that a component is formed only of the element(s) listed.

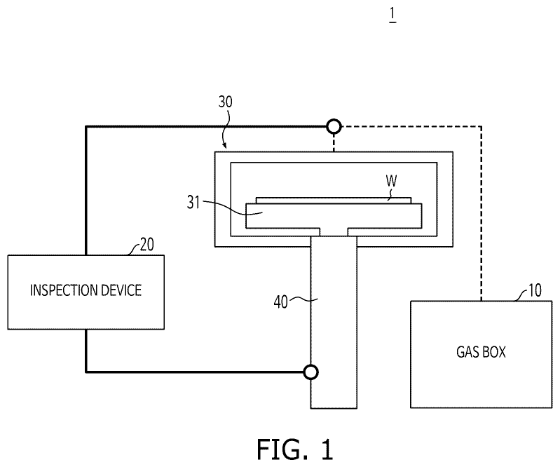

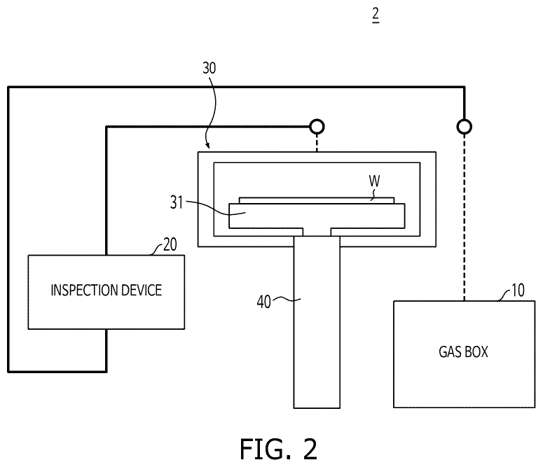

FIGS. 1 and 2 illustrate a substrate processing apparatus according to some embodiments of the present disclosure.

Referring to FIGS. 1 and 2, a substrate processing apparatus 1 according to some embodiments of the present disclosure may include a gas box 10, an inspection device 20, a chamber 30, and a foreline 40. As used herein, the term “gas box” need not be an actual box in shape or configuration.

The substrate processing apparatus 1 may process a substrate. The substrate processing apparatus 1 may be a deposition apparatus that forms a thin film on a substrate W provided on a substrate support 31 inside the chamber 30. For example, a deposition process may include a physical vapor deposition (PVD), a chemical vapor deposition (CVP), or an atomic layer deposition (ALD) method. In some embodiments, the substrate processing apparatus 1 may be an apparatus that is able to perform an etching process, a photolithography process, an ion implantation process, an annealing process, and the like, but the present disclosure is not limited thereto. The substrate processing apparatus 1 may also perform another process using gas. The substrate W may be a silicon wafer used in manufacturing a semiconductor device. The semiconductor device may be for example, a semiconductor chip (i.e., a semiconductor device singulated from (e.g., cut from) a wafer).

The gas box 10 may be configured to store gas. For example, the gas box 10 may store a process gas for processing the substrate W. The process gas may be supplied to the chamber 30. For example, the process gas may include various gases such as SiH₄, SiCl₄, NH₃, and/or the like.

The inspection device 20 may be configured to be supplied with the gas from the gas box 10 and inspect the gas. The inspection device 20 may detect residual gas, particles, or the like present in the process gas stored in the gas box 10. The residual gas may be a remaining process gas and may include a toxic gas such as SiH₄.

The inspection device 20 may be supplied with the gas from the gas box 10. The gas stored in the gas box 10 may be discharged to the chamber 30 through the inspection device 20. In some embodiments, the gas stored in the gas box 10 may be discharged to the foreline 40 through the inspection device 20.

The chamber 30 may provide a room for processing the substrate W. The substrate support 31 may be provided inside the chamber 30. The substrate W may be set safely on the substrate support 31. The substrate W may be processed using the process gas supplied to the chamber 30. The atmosphere of the internal room of the chamber 30 may be controlled to a vacuum state, or a pressure of the internal room may be controlled to a pressure level. For example, the internal room of the chamber 30 may be in a vacuum state.

The chamber 30 may be provided with the foreline 40 and an exhaust pump (not illustrated in the drawing). The foreline 40 may be provided at a lower portion of the chamber 30. One end of the foreline 40 may be connected to the chamber 30, and the other end of the foreline 40 may be connected to the exhaust pump. The exhaust pump may be a vacuum pump such as a turbo molecular pump. The exhaust pump may control a vacuum level of the room inside the chamber 30. Process by-products and residual process gases generated in the chamber 30 may be discharged through the foreline 40.

In an embodiment, referring to FIG. 1, the gas box 10 may supply gas to the inspection device 20. The inspection device 20 may inspect whether residual gas or particles are present in the gas supplied from the gas box 10, and then discharge the gas through the foreline 40.

In another embodiment, referring to FIG. 2, the gas box 10 may supply gas to the inspection device 20. The inspection device 20 may inspect whether residual gas or particles are present in the gas supplied from the gas box 10, and then discharge the gas into the chamber 30. The gas discharged into the chamber 30 may be discharged together in discharging the gas inside the chamber 30. For example, the gas discharged from the inspection device 20 to the chamber 30 may be discharged to the outside again through the foreline 40.

The inspection device 20 according to some embodiments of the present disclosure may be connected to at least one of the chamber 30 and/or the foreline 40. The inspection device 20 may be selectively connected to the chamber 30 or the foreline 40. In some embodiments, the inspection device 20 may be connected to both of the chamber 30 and the foreline 40. In such a manner, the inspection device 20 is connectable to the chamber 30 or the foreline 40. Therefore, the inspection device 20 may be used to detect residual gas, particles, or the like present in the process gas stored in the gas box 10 under various conditions of the process performed by the chamber 30.

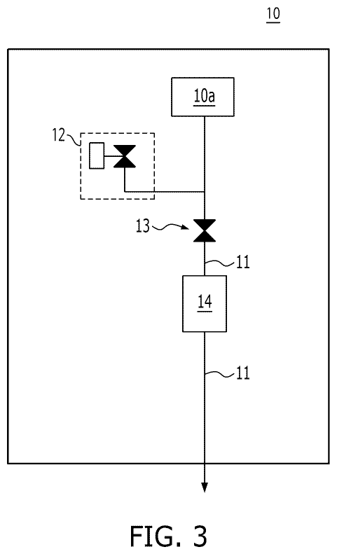

FIG. 3 illustrates a configuration of a gas box according to some embodiments of the present disclosure.

Referring to FIG. 3, the gas box 10 according to some embodiments of the present disclosure may include a gas line 11, a purge supply 12, a gas valve 13, and a flow controller 14.

The gas line 11 may be configured to allow the gas to move therethrough. The gas line 11 may be connected to a gas storage container 10a (such as a gas canister or gas cylinder, for example) to be supplied with the gas. The gas line 11 may extend along one direction to allow the supplied gas to move. The gas line 11 may be connected to a chamber 30 to supply the gas moved through the inside thereof.

The purge supply 12 may be connected to the gas line 11. The purge supply 12 may supply purge gas to the gas line 11. For example, the purge gas may include nitrogen (N2) gas, argon (Ar) gas, and/or the like. The purge supply 12 may purge the gas line 11. For example, the purge supply 12 may supply nitrogen gas to the gas line 11 at about 10,000 sccm, or about 8000 to about 12000 sccm, or about 9000 to about 11000 sccm.

The gas valve 13 may selectively open and close the gas line 11. The gas valve 13 may restrict the movement of gas moving along the gas line 11. The gas valve 13 may be disposed downstream of the purge supply 12.

In example embodiments, a plurality of gas valves 13 may be provided. For example, the gas valve 13 may further include an upstream valve positioned on the upstream side of the flow controller 14, a downstream valve positioned on the downstream side of the flow controller 14, and/or a final valve positioned at an even more downstream side of the gas box 10 (such as near an exit from the gas box 10), and the like.

The flow controller 14 may control a flow rate of the gas moving along through the gas line 11. For example, the flow controller 14 may be a mass flow controller (MFC). The flow controller 14 may selectively open and close the gas line 11. The flow controller 14 may close the gas line 11 by setting the flow rate of the gas moving along the gas line 11 to 0%. The flow controller 14 may completely open the gas line 11 by setting the flow rate of the gas moving along the gas line 11 to 100%. However, the present disclosure is not limited thereto, and the flow controller 14 may open only a part of the gas line 11 by setting the flow rate of the gas moving along the gas line 11 to 50% or the like, or to any amount between 0 and 100%.

The purge supply 12 may be disposed upstream of the gas line 11. For example, the purge supply 12 may be disposed upstream of the gas valve 13 and the flow controller 14. In the gas box 10 according to some embodiments of the present disclosure, the gas valve 13 may be disposed upstream of the flow controller 14. However, the present disclosure is not limited thereto, and in example embodiments, the flow controller 14 may also be disposed upstream of the gas valve 13.

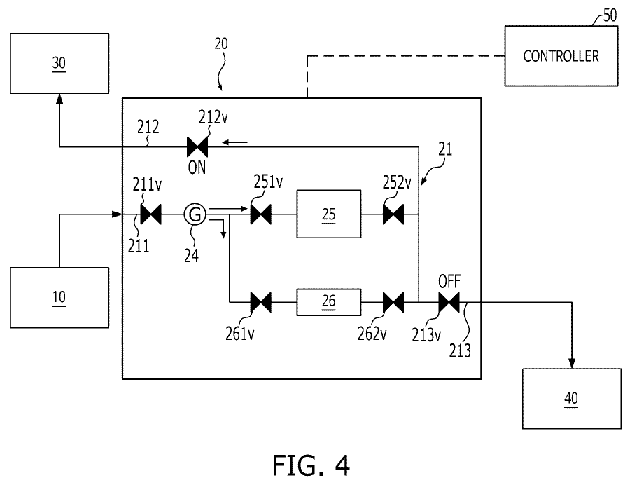

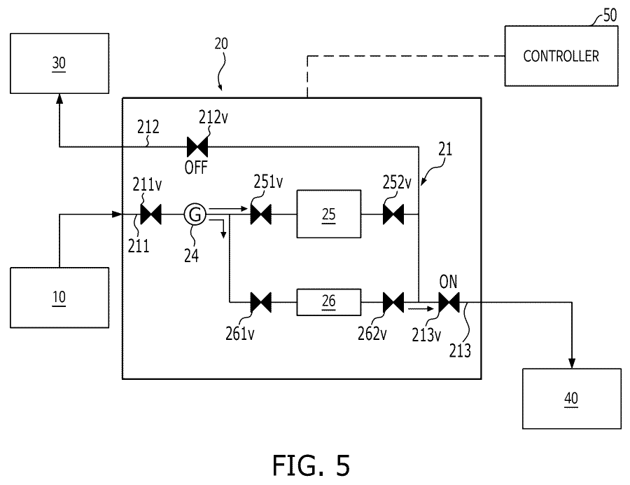

FIGS. 4 and 5 illustrate a configuration of an inspection system according to some embodiments of the present disclosure.

Referring to FIGS. 4 and 5, the inspection system according to some embodiments of the present disclosure may include the gas box 10, the inspection device 20, the chamber 30, the foreline 40, and a controller 50. In these embodiments, the configuration of the gas box 10 may be the same as the configuration of the gas box 10 of FIG. 3.

In example embodiments, the gas box 10 may be connected to the inspection device 20. The gas discharged from the gas box 10 may be introduced into the inspection device 20. The gas box 10 may supply the gas to the inspection device 20. For example, the gas line 11 may extend to an inspection line 21 that extends through the inspection device 20. The gas line 11 and the inspection line 21 may be connected at the gas box 10, at the inspection device 20 or at a location therebetween. The gas line 11 and inspection line 21 may independently be for example a tube, a pipe or any acceptable conduit that allows gas to flow therethrough, and may be connected by an acceptable attachment that allows gas to flow from the gas line 11 to the inspection line 21. According to non-limiting examples, the gas line 11 may be detachably attached. When gas flows from the gas box 10 to the inspection device 20, the inspection device 20 may inspect whether or not the inside of the gas box 10 is contaminated. In some embodiments, the inspection device 20 may inspect whether or not the gas line 11, the gas valve 13, the flow controller 14, and the like constituting the gas box 10 are contaminated. For example, the inspection device 20 may inspect a presence or absence of residual gas or particles inside the gas supplied from the gas box 10.

The inspection device 20 may be configured to be connectable to at least one of the chamber 30 and/or the foreline 40. The inspection device 20 may be configured to be easily connected to and separated from the gas box 10 so as to inspect whether or not a plurality of gas boxes 10 are contaminated. For example, the inspection device 20 may be connected to a first gas box 10 to inspect whether or not the first gas box 10 is contaminated, and then disconnected from the first gas box 10, so that the inspection device 20 may be connected to a second gas box 10 to inspect whether or not the second gas box 10 is contaminated, etc. The inspection device 20 may be configured to be connected to and separated from the gas box 10 for example in connecting the gas line 11 to the inspection line 21, or one of those lines may be connected to an outlet or inlet of the other line, and/or there may be other or additional connections between the gas box 10 and the inspection device 20. In some embodiments, for the configuration, the inspection device 20 may be configured to easily move. For example, the inspection device 20 may be provided with wheels to move. According to example embodiments, the inspection device 20 may be movable from one gas box 10 to another gas box 10 when the inspection device 20 is disconnected from a gas box 10.

The inspection device 20 may be connected to the chamber 30. The gas passing through the inspection device 20 via the inspection line 21 may be provided to the chamber 30. The gas passing through the inspection device 20 may contain residual gas or particles. Therefore, the gas may be discharged through the chamber 30 and through the foreline 40.

The inspection device 20 may be connected to the foreline 40. The gas passing through the inspection device 20 via the inspection line 21 may be discharged through the foreline 40. The gas passing through the inspection device 20 may contain residual gas or particles. Therefore, the gas may be discharged through the foreline 40.

The inspection device 20 may include an inspection line 21, a pressure gauge 24, a first sensor 25, and a second sensor 26. In example embodiments, the inspection line 21 may extend beyond the inspection device 20 toward one or more of the gas box 10, the chamber 30 and/or the foreline 40.

In example embodiments, the inspection line 21 may include an inlet port 211 and an inlet valve 211v at an inlet to the inspection device 20, and outlet ports 212 and 213 and outlet valves 212v and 213v at outlets from the inspection device 20.

The inlet port 211 may be connected to the gas box 10 to be supplied with the gas from the gas box 10. The gas discharged from the gas box 10 may be supplied to the inspection device 20 through the inlet port 211. The inlet valve 211v may be configured to selectively open and close the inlet port 211. The inlet valve 211v may be disposed upstream of the inspection line 21. For example, the inlet valve 211v may control the flow of gas supplied to the inspection device 20.

The outlet ports 212 and 213 may include a first outlet port 212 and a second outlet port 213. The first outlet port 212 may be connected to the chamber 30 such that the gas passing through the inspection line 21 is discharged to the chamber 30. The second outlet port 213 may be connected to the foreline 40 such that the gas is discharged to the foreline 40. The outlet valves 212v and 213v may include a first outlet valve 212v and a second outlet valve 213v. The first outlet valve 212v may selectively open and close the first outlet port 212. The second outlet valve 213v may selectively open and close the second outlet port 213.

The inspection device 20 may discharge the gas into the chamber 30 by opening the first outlet valve 212v and closing the second outlet valve 213v while being connected to each of the chamber 30 and the foreline 40. In some embodiments, the inspection device 20 may discharge the gas into the foreline 40 by closing the first outlet valve 212v and opening the second outlet valve 213v while being connected to each of the chamber 30 and the foreline 40.

A pressure gauge 24 may be provided on the inspection line 21. The pressure gauge 24 may measure a pressure of the gas flowing along the inspection line 21. The pressure gauge 24 may display the pressure of the gas within the inspection line 21. According to example embodiments, the pressure gauge may transmit a pressure value to another device from which pressure of gas within the inspection line 21 may be determined. The pressure gauge 24 may be positioned downstream of the inlet valve 212v, but the present disclosure is not limited thereto.

The first sensor 25 may be a particle sensor. The first sensor 25 may detect solid-state fine particles contained within the gas. The first sensor 25 may detect the number of particles. For example, the first sensor 25 may detect solid-state particles fixed inside the gas box 10 (for example, the gas valve 13 or the flow controller 14).

The second sensor 26 may be a gas sensor. The second sensor 26 may detect a target gas contained in the gas. The second sensor 26 may detect for example, a precursor gas or a toxic gas remaining in the gas box 10. For example, the second sensor 26 may detect SiH₄ (Silane) which is a toxic gas.

According to example embodiments, the first sensor 25 and the second sensor 26 may be disposed in parallel with each other. For example, the inspection line 21 may be branched upstream of the first sensor 25 and the second sensor 26. The gas moving along the inspection line 21 may be branched and flow to each of the first sensor 25 and the second sensor 26.

Each of the first sensor 25 and the second sensor 26 may detect whether or not the gas box 10 is contaminated. For example, the first sensor 25 may detect whether or not particles are present in the gas flowing from the gas box 10, and the second sensor 26 may detect whether or not the target gas is present in the gas flowing from the gas box 10. In some embodiments, one sensor may detect both particles and target gas. In a case where either of the first sensor 25 and the second sensor 26 detects particles or target gas in the gas, the gas box 10 may be contaminated. The gas, which is branched by the inspection line branching along the lines where the first sensor 25 and the second sensor 26 are respectively provided on the inspection line 21, may be joined again downstream of the first sensor 25 and the second sensor 26, and be discharged to the chamber 30 and/or the foreline 40.

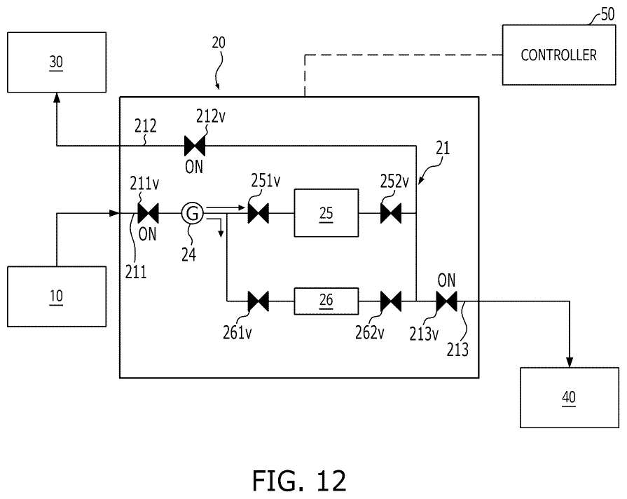

In examples in which the inspection line 21 is branched prior to the first sensor 25 and the second sensor 26, the inspection line 21 may be provided with first sensor valves 251v and 252v, which are respectively disposed upstream and downstream of the first sensor 25 (a first first sensor valve 251v upstream from the first sensor and a first second sensor valve 252v downstream from the first sensor), and second sensor valves 261v and 262v, which are respectively disposed upstream and downstream of the second sensor 26 (a second first sensor valve 261v upstream from the first sensor and a second second sensor valve 262v downstream from the first sensor). The first sensor valves 251v and 252v make it possible for the first sensor 25 to be separated from the inspection line 21 for replacement or repair of the first sensor 25. Before separating or replacing the first sensor 25, the first sensor valves 251v and 252v may be closed. the second sensor valves 261v and 262v make it possible for the second sensor 26 to be separated from the inspection line 21 for replacement or repair of the second sensor 26. Before separating or replacing the second sensor 26, the second sensor valves 261v and 262v may be closed. In replacing the first sensor 25 or the second sensor 26, outside air may be introduced due to a defect in the connection portion or the like. Whether or not outside air is introduced into the inspection line 21 may be checked through the pressure gauge 24. A detailed description thereof will be described with reference to FIGS. 12 to 14.

To remove particles or residual gas remaining in the inspection line 21, purge gas may be supplied from the gas box 10. For example, 10,000 sccm, or 8,000 sccm to 12,000 sccm, or 9,000 sccm to 11,000 sccm of nitrogen (N2) gas may be supplied from the gas box 10. The purge gas introduced into the inspection line 21 may be discharged through the outlet valves 212v and 213v. In a case where the inspection device 20 is connected to the chamber 30, the purge gas may be discharged into the chamber 30 while the first outlet valve 212v is repeatedly opened and closed. In some embodiments, in a case where the inspection device 20 is connected to the foreline 40, the purge gas may be discharged to the foreline 40 while the second outlet valve 213v is repeatedly opened and closed.

For example, in FIG. 4, after the second outlet valve 213v is closed and all the remaining valves are opened, the purge gas may be supplied from the gas box 10 through the inspection line 21. Then, while the second outlet valve 213v is repeatedly opened and closed at a predetermined time interval, the purge gas may be discharged to the chamber 30. In FIG. 5, after the first outlet valve 212v is closed and all the remaining valves are opened, the purge gas may be supplied from the gas box 10 through the inspection line 21. Then, while the first outlet valve 212v is repeatedly opened and closed at a predetermined time interval, the purge gas may be discharged to the foreline 40.

The controller 50 may control at least one of the gas box 10 and/or the inspection device 20. The controller 50 may control the gas box 10 to determine which component(s) among the gas line 11, the gas valve 13, the flow controller 14, and the like of the gas box 10 is contaminated. The controller 50 may control the inlet valve 211v, the outlet valves 212v and 213v, the first sensor valves 251v and 252v, the second sensor valves 261v and 262v, and the like of the inspection device 20.

In example embodiments, examples of controlling the component of the inspection system through the controller 50 may include any one or all of directly transmitting a control signal to the corresponding component, transmitting a control signal to a separate driving device that drives the corresponding component, and transmitting a control signal to another intermediate component necessary to control the corresponding component.

The controller 50 may include: a memory that stores various data and a program for executing the operations described above and operations to be described later; and a processor that processes the data by executing the program stored in the memory. Such a controller 50 may be formed by several interconnected controllers and may be configured by software.

The memory may include at least one of volatile memories such as a static random access memory (SRAM), a dynamic random access memory (DRAM), and nonvolatile memories such as a flash memory, a read only memory (ROM), an erasable programmable read only memory (EPROM), and/or an electrically erasable programmable read only memory (EEPROM).

The nonvolatile memory may operate as an auxiliary memory device of the volatile memory, and may maintain stored data even in a case where the power of the plasma etching device is cut off. For example, the nonvolatile memory may store control data and a control program for controlling an operation of the plasma etching device or an operation of the gas cooling device.

Unlike the nonvolatile memory, the volatile memory may lose stored data in a case where the power of the plasma etching device is cut off. The volatile memory may load the control program and the control data from nonvolatile memory and temporarily store the control program and the control data, temporarily store input setting or a control command, or temporarily store a control signal which is output from the processor.

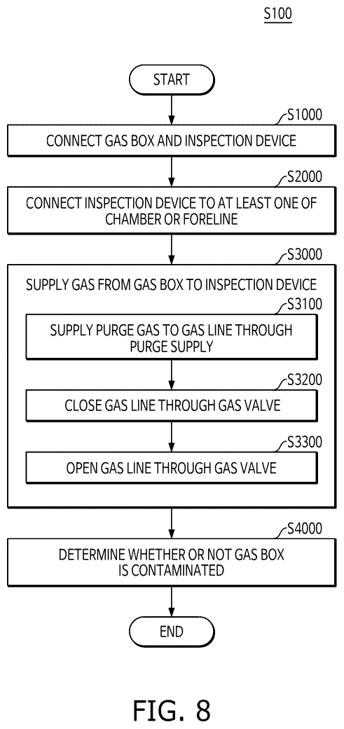

The processor (i.e., a hardware circuit), may be a microprocessor, a CPU (Central Processing Unit), a GPU (graphics processor), a digital signal processor (DSP), a field-programmable gate array (FPGA), etc., and may be part of a computer. The computer may be a general purpose computer or may be dedicated hardware or firmware (e.g., an electronic or optical circuit, such as application-specific hardware, such as, for example, a digital signal processor (DSP) or a field-programmable gate array (FPGA)). A computer may be configured from several interconnected computers. The processor may process data or output control signals in accordance with the program stored in the memory. For example, the processor may process data or output control signals in accordance with a program which includes commands for executing the inspection method described with reference to FIGS. 8 to 10 and FIGS. 14 and 16 stored in the memory.

The processor and the memory may be provided as a single component or a plurality of components depending on capacities thereof. In some embodiments, the processor and the memory may be provided to be physically separated from each other or may be provided as a single chip.

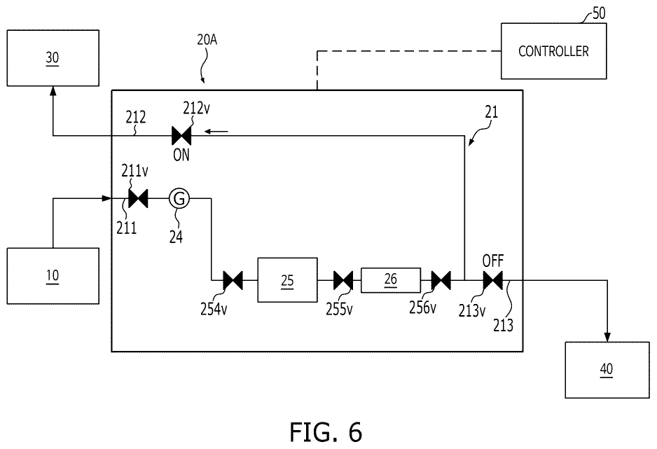

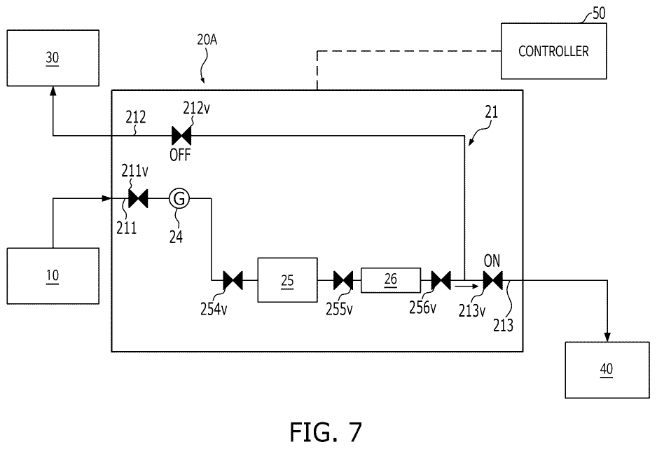

FIGS. 6 and 7 illustrate a configuration of an inspection system according to some embodiments of the present disclosure. For convenience of explanation, the same components as those of the embodiments of FIGS. 4 and 5 are represented by the same reference numerals and signs, and detailed descriptions thereof may not be repeated.

Referring to FIGS. 6 and 7, an inspection system according to some embodiments of the present disclosure may include an inspection device 20A.

The inspection device 20A may include the first sensor 25 and the second sensor 26 that are disposed on the inspection line 21. In these example embodiments, the first sensor 25 and the second sensor 26 may be disposed in series with each other. The first sensor 25 may be disposed upstream of the second sensor 26. In some embodiments, the second sensor 26 may be disposed upstream of the first sensor 25.

The gas passing through the first sensor 25 on the inspection line 21 may flow to the second sensor 26. Each of the first sensor 25 and the second sensor 26 may detect whether or not the gas box 10 is contaminated. For example, the first sensor 25 may detect whether or not particles are present in the gas flowing from the gas box 10, and the second sensor 26 may detect whether or not the target gas is present in the gas flowing. In a case where one or both of the first sensor 25 and/or the second sensor 26 detects particles or target gases in the gas, it may be determined that the gas box 10 is contaminated.

In example embodiments, the inspection line 21 may be provided with a first sensor valve 254v disposed upstream of the first sensor 25, a second sensor valve 255v disposed between the first sensor 25 and the second sensor 26, and a third sensor valve 256v disposed downstream of the second sensor 26. The first sensor 25 may be separated from the inspection line 21 for replacement or repair of the first sensor 25. Before separating the first sensor 25, the first sensor valve 254v and the second sensor valve 255v may be closed. In some embodiments, to replace or repair the second sensor 26, the second sensor 26 may be separated from the inspection line 21. Before replacing the second sensor 26, the second sensor valve 255v and the third sensor valve 256v may be closed. Outside air may be introduced due to replacement of the first sensor 25 or the second sensor 26, a defect in the connection portion of the sensors, or the like. Whether or not outside air is introduced into the inspection line 21 may be checked through the pressure gauge 24. A further description thereof will be described with reference to FIGS. 12 to 14.

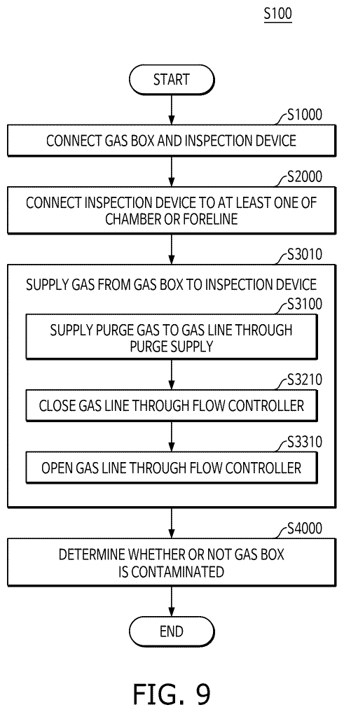

FIGS. 8 and 9 illustrate flow charts showing inspection methods according to some embodiments of the present disclosure.

Referring to FIG. 8, an inspection method S100 according to some embodiments of the present disclosure may include first to fourth steps S1000 to S4000.

In a case where the method according to some embodiments may be implemented in a different way, specific steps may be performed in a different order from the described order. For example, two successive steps described may be performed substantially simultaneously or may be performed in a reverse order of the described order.

An inspection method S100 according to some embodiments of the present disclosure may include Step S1000 of connecting the gas box and the inspection device.

The upstream side of the inspection device may be connected to the downstream side of the gas box. For example, an inlet port of the inspection device may be connected to the gas box, such as connecting a gas line of the gas box to the inlet port of the inspection line. The gas box may supply gas to the inspection device connected to the gas box.

The inspection method S100 according to some embodiments of the present disclosure may include Step S2000 of connecting the inspection device with at least one of the chamber or the foreline.

The downstream side of the inspection device may be connected to the chamber or the foreline. For example, the inlet port of the inspection line in the inspection device may be connected to the gas box. The inspection device may be supplied with the gas from the gas box to perform the inspection, and may discharge the gas into the chamber or the foreline.

The inspection line within the inspection device may be physically connected to the chamber and the foreline. For example, the first outlet port of the inspection line may be connected to the chamber, and the second outlet port may be connected to the foreline. In such a configuration, the first outlet valve may be opened and the second outlet valve may be closed, and thus the gas may not be discharged to the foreline and may be discharged to the chamber. Conversely, the first outlet valve may be closed and the second outlet valve may be opened, and thus the gas may not be discharged to the chamber and may be discharged to the foreline.

In some embodiments, the inspection line may be physically connected to the chamber and not connected to the foreline. In other embodiments, the inspection line may be physically connected to the foreline and not connected to the chamber. In other embodiments, the inspection line may be physically connected to both the chamber and the foreline, for example if the inspection line is branched.

The inspection method S100 according to some embodiments of the present disclosure may include Step S3000 of supplying the gas from the gas box to the inspection device.

Step S3000 of supplying gas to the inspection device may include Step S3100 of supplying purge gas to the gas line through the purge supply. In a case of supplying the purge gas, all gas valves provided in the gas line may be being opened. The purge supply may be disposed upstream of the gas valve inside the gas box. The purge supply may supply the purge gas to the gas line. For example, the purge supply may supply nitrogen gas at 10,000 sccm, or about 8000 to about 12000 sccm, or about 9000 to about 11000 sccm for a predetermined time. In such a case, the purge gas may be provided to have an amount sufficient to saturate the gas line.

Step S3000 of supplying gas to the inspection device may include Step S3200 of closing the gas line through the gas valve. The gas valve may close the gas line to restrict the movement of gas in the gas box. The gas may move from the upstream side of the gas valve to the gas valve, and may not move downstream through the closed gas valve. For example, the purge gas may be trapped in a high pressure state on the upstream side of the gas valve in the gas line. Therefore, a high pressure may be formed upstream of the gas line. The gas may be discharged from the inspection device through the chamber or the foreline. Therefore, a relatively low pressure may be formed downstream of the gas line. For example, a pressure difference may be formed between the upstream side and the downstream side of the gas valve in the gas line.

Step S3000 of supplying gas to the inspection device may include Step S3300 of opening the gas line through the gas valve. The gas valve may be opened in a state where the gas line is closed using the gas valve. For example, the gas valve may be switched to open the gas line. The purge gas trapped in a high pressure state through the gas valve may pass through the gas valve and move along the gas line. The purge gas may carry residual gas, particles, or the like remaining in the gas valve while passing through the gas valve. The purge gas may carry residual gas, particles, or the like remaining in the gas line to the inspection device.

The inspection method S100 according to some embodiments of the present disclosure may include Step S4000 of determining whether or not the gas box is contaminated. In a case where residual gas or particles carried by the purge gas are present, the first sensor and/or the second sensor provided in the inspection device may detect the residual gas or particles. For example, in a case where the particle sensor detects particles carried by the purge gas, the particle sensor may output data indicating that particles are detected. The particle sensor may transmit the output data to the controller. In a case where the gas sensor detects the residual gas (for example, precursor gas or toxic gas) carried by the purge gas, the gas sensor may output output data indicating that the residual gas is detected. The gas sensor may transmit the output data to the controller.

The controller may determine whether or not the gas box is contaminated on the basis of the output data which is output from at least one of the first sensor or the second sensor. In a case where the first sensor detects particles and/or the second sensor detects gas, the controller may determine that the gas box is contaminated. In such a case, the controller may determine that the gas valve is contaminated. In an event that contamination of the gas box or any other component (or the lack of contamination) is determined by the controller, the controller may provide a display, sound, light, text, or any other alert to communicate that there is contamination. In addition, or alternatively, in an event that contamination of the gas box or any other component (or lack of contamination) is determined by the controller, the controller may control the flow of gas e.g. by opening or closing of valves. The controller may also store data regarding detection of contamination (or lack of contamination).

Referring to FIG. 9, an inspection method S100 according to some embodiments of the present disclosure may include first to fourth steps S1000 to S4000. For convenience of explanation, only steps different from the inspection method of FIG. 8 will be described.

The inspection method S100 according to some embodiments of the present disclosure may include Step S3010 of supplying the gas from the gas box to the inspection device.

Step S3010 of supplying gas to the inspection device may include Step S3100 of supplying purge gas to the gas line through the purge supply of the gas box. In a case of supplying the purge gas, all gas valves provided in the gas line may be being opened. The purge supply may be disposed upstream of the flow controller in the gas box. The purge supply may supply the purge gas to the gas line. For example, the purge supply may supply nitrogen gas at 10,000 sccm, or about 8000 to about 12000 sccm, or about 9000 to about 11000 sccm for a predetermined time. In such a case, the purge gas may be provided to have an amount sufficient to saturate the gas line.

Step S3010 of supplying gas to the inspection device may include Step S3210 of closing the gas line through the flow controller. The flow controller may close the gas line in a state where the flow controller opens the gas line, thereby restricting the movement of the gas. The gas may be moved from the upstream side of the flow controller to the flow controller in the gas line. For example, the gas may be moved through the gas line located upstream of the gas valve and the flow controller. The gas may not be moved downstream of the flow controller. For example, the purge gas may be trapped in a high pressure state on the upstream side of the flow controller in the gas line. Therefore, a high pressure may be formed upstream of the flow controller. The gas may be discharged from the inspection device through the chamber or the foreline. Therefore, a relatively low pressure may be formed downstream of the gas line. For example, a pressure difference may be formed between the upstream side and the downstream side of the flow controller.

Step S3010 of supplying gas to the inspection device may include Step S3310 of opening the gas line through the flow controller. The flow controller may be opened in a state where the gas line is closed. For example, the flow controller may control the flow rate of the gas flowing along the gas line to 100% by opening the gas line. The purge gas, which is trapped in a high pressure state by the closed flow controller on the gas line, may pass through the open flow controller and move along the gas line. The purge gas may carry residual gas, particles, or the like remaining in the flow controller while passing through the flow controller. The purge gas may carry residual gas, particles, or the like remaining in the gas line to the inspection device.

The inspection method (S100) according to some embodiments of the present disclosure may include Step S4000 of determining whether or not the gas box is contaminated. In a case where residual gas or particles carried by the purge gas are present, the first sensor or the second sensor provided in the inspection device may detect the residual gas or particles and may output data indicating that the residual gas or particles are detected. The first sensor or the second sensor may transmit the output data to the controller.

The controller may determine whether or not the gas box is contaminated on the basis of the output data which is output from at least one of the first sensor or the second sensor. In a case where the first sensor detects a particle or the second sensor detects gas, the controller may determine that the gas box is contaminated. For example, the inspection device may determine that the flow controller is contaminated.

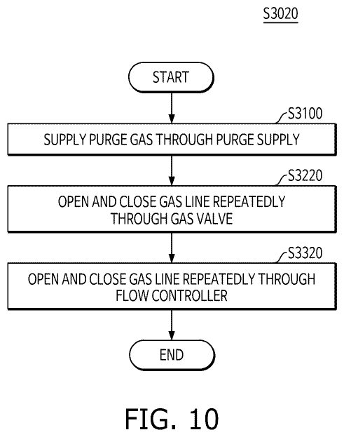

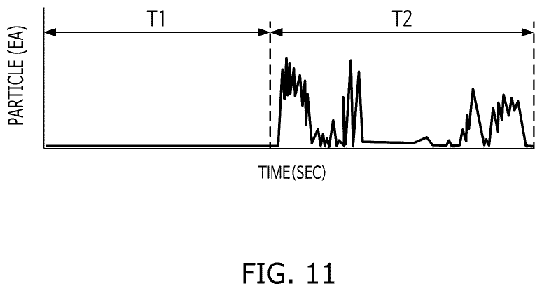

FIG. 10 illustrates a flow chart showing an inspection method according to some embodiments of the present disclosure. FIG. 11 illustrates a graph showing a result according to an inspection method according to some embodiments of the present disclosure. For the convenience of explanation, detailed descriptions of steps the same as the steps in FIGS. 8 and 9 may not be repeated.

Referring to FIG. 10, Step S3020 of supplying gas from the gas box to the inspection device may include the first to third steps S3100, S3220, and S3320. For example, in the method S100 of FIG. 9, Step S3020 may be executed instead of Step S3010.

Step S3020 of supplying gas from the gas box to the inspection device may include Step S3320 of repeatedly opening and closing the gas line through the gas valve.

In a case where the gas valve disposed in the gas box is opened and closed, the purge gas may pass through the gas valve at a high speed due to the pressure difference. For example, the gas valve may be repeatedly opened and closed 10 times for about 100 seconds, or opened and closed 8-12 times for 80-120 seconds. By repeating the opening and closing, contaminated particles or residual gas on the inside of the gas valve and the wall surface of the gas pipe may be cleaned. The purge gas may carry the particles or residual gas to the inspection device.

Step S3020 of supplying gas from the gas box to the inspection device may include Step S3330 of repeatedly opening and closing the gas line through the flow controller.

The purge gas may be trapped in a high-pressure state on the upstream side of the flow controller. Thus, a relatively large pressure difference may occur between the upstream side and the downstream side of the gas valve. In a case where the gas valve is opened and closed, the purge gas may pass through the gas valve at a high speed due to the pressure difference. For example, the flow controller may be repeatedly opened and closed 10 times for about 200 seconds, or opened and closed 8-12 times for 180-220 seconds. By repeating the opening and closing, contaminated particles or residual gas on the inside of the flow controller and the wall surface of the gas pipe may be cleaned. The purge gas may carry the particles or residual gas to the inspection device.

Referring to FIG. 11, the X-axis of the graph may represent the time (seconds), and the Y-axis of the graph may represent the particles (number). FIG. 11 is a graph illustrating an example of the result in a case where Step S3020 of FIG. 10 is performed.

A first time T1 may correspond to Step S3220 of repeatedly opening and closing the gas valve. A second time T2 may correspond to Step S3320 of repeatedly opening and closing of the flow controller. Because the number of particles detected by the inspection device at the first time T1 does not increase and is close to 0, it may be determined that the gas valve is not contaminated. In contrast, because the number of particles detected by the inspection device at the second time T2 rapidly increases and a large number of particles are detected, it may be determined that the flow controller is contaminated. In such a case, only the flow controller may be repaired or replaced without replacing the entire gas box determined to be contaminated.

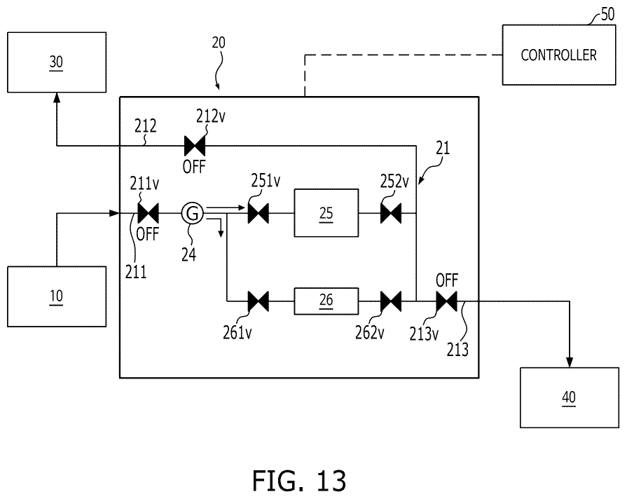

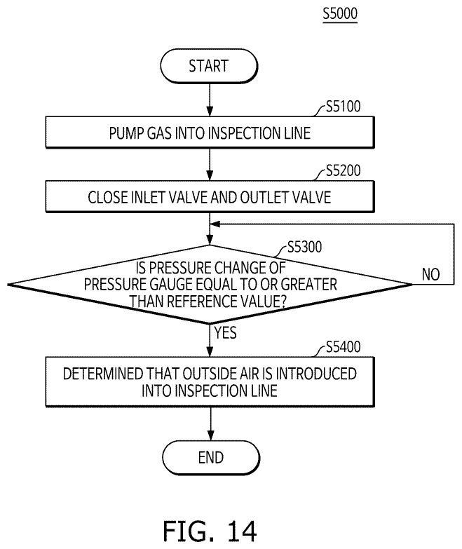

FIGS. 12 and 13 illustrate diagrams for explaining an inspection system according to some embodiments of the present disclosure. FIG. 14 illustrates a flow chart showing an inspection method according to some embodiments of the present disclosure. For convenience of explanation, the same components as those of the embodiments of FIGS. 4 and 5 are represented by the same reference numerals and signs, and detailed descriptions thereof may not be repeated.

Referring to FIGS. 12 and 13, the first sensor 25 or the second sensor 26 may be separated from the inspection line 21 to replace or repair the first sensor 25 or the second sensor 26.

In a case of separating the first sensor 25 therefrom, the first sensor valves 251v and 252v may be first closed. In some embodiments, in a case of replacing the second sensor 26, the second sensor valves 261v and 262v may be first closed. In such a case, outside air may be introduced due to a defect in the connection portion and the like in a case of mounting the first sensor 25 or the second sensor 26. Whether or not outside air is introduced into the inspection line 21 may be checked through the pressure gauge 24.

For example, the connection portion of the first sensor 25 or the connection portion of the second sensor 26 may be loosely connected or incorrectly connected in a case of mounting the first sensor 25 or the second sensor 26. In such a case, outside air may be introduced through the connection portion of the first sensor 25 or the connection portion of the second sensor 26, which may be checked by the pressure gauge 24.

Referring to FIGS. 12 and 14, an inspection method (S5000) according to some embodiments of the present disclosure may include Step S5100 of pumping gas into the inspection line.

The gas may be pumped and injected into the inspection line 21. In such a case, all valves disposed in the inspection line 21 may be being opened. For example, the inlet valve 211v, the outlet valves 212v and 213v, the first sensor valves 251v and 252v, and the second sensor valves 261v and 262v all may be being opened. The gas injected into the inspection line 21 may saturate the inside of the inspection line 21. The gas may be injected for a sufficient time to keep the inside of the inspection line 21 saturated. For example, the gas pumping may be performed for about 10 minutes, or 8-12 minutes.

Referring to FIG. 13, the inspection method S5000 according to some embodiments of the present disclosure may include Step S5200 of closing the inlet valve and the outlet valve. After gas is injected into the inspection line 21, the inlet valve 211v and the outlet valves 212v and 213v may be closed. For example, all the inlet valve 211v, the first outlet valve 212v, and the second outlet valve 213v may be closed. Consequently, the gas injected into the inspection line 21 may be trapped inside the inspection line 21 within the inspection device, as the inlet valve 211v and the outlet valves 212v and 213v are closed.

The gas inside the inspection line 21 may have a predetermined pressure value. The pressure inside the inspection line 21 may be referred to as an initial pressure. In a case where outside air is introduced into the inspection line 21, the internal pressure of the inspection line 21 may increase. Consequently, the pressure gauge 24 may measure a pressure that changes as outside air is introduced into the inspection line 21.

The inspection method (S5000) according to some embodiments of the present disclosure may include Step S5300 of determining whether the pressure change of the pressure gauge is equal to or greater than a reference value.

In a case where outside air is introduced into the inspection line 21, the pressure value of the pressure gauge 24 may increase. The pressure gauge 24 may calculate a difference between an initial pressure value and an increased pressure value. Consequently, a pressure change value of the pressure gauge 24 may be calculated.

It may be determined whether the pressure change value calculated by the pressure gauge 24 is equal to or greater than a reference value. The reference value may be a preset value. The preset value may be approximately 1 mTorr/minute. For example, in a case where the pressure change value of the pressure gauge 24 measured for about 5 minutes is equal to or greater than 5 mTorr, it can be determined that the pressure change value is equal to or greater than the reference value. In other embodiments, the reference value may be set to a different value, such as 4-6 mTorr.

The inspection method (S5000) according to some embodiments of the present disclosure may include Step S5400 of determining whether outside air is introduced into the inspection line.

On the basis of the measured value of the pressure gauge 24, it may be determined whether or not outside air is introduced. In a case where the pressure change value calculated by the pressure gauge 24 is equal to or greater than a reference value, it can be determined that outside air is introduced. In a case where the pressure change value calculated by the pressure gauge 24 is less than the reference value, it can be determined that outside air is not introduced.

In some embodiments, the pressure gauge 24 may transmit the pressure change value to the controller 50. The controller 50 may compare the pressure change sent from the pressure gauge 24 with the reference value to determine whether or not outside air is introduced.

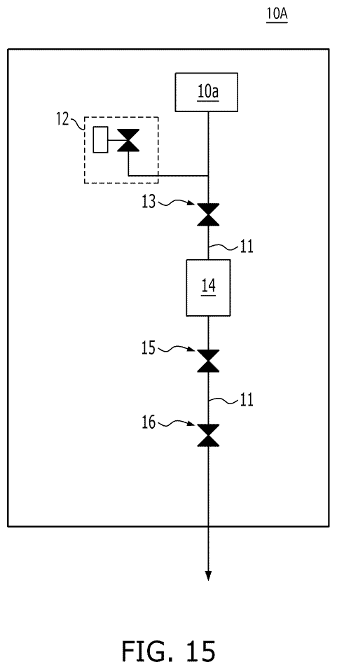

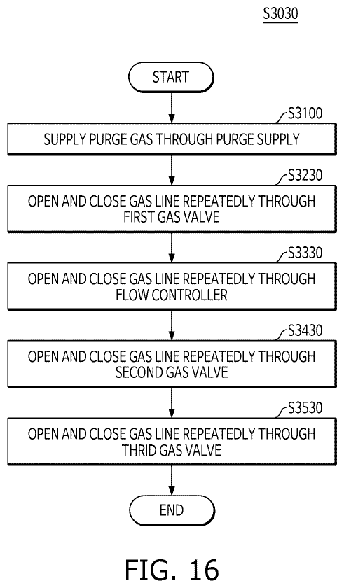

FIG. 15 illustrates a diagram for explaining a gas box according to some embodiments of the present disclosure. FIG. 16 illustrates a flow chart showing an inspection method according to some embodiments of the present disclosure. For convenience of explanation, the same components as those of the embodiments of FIGS. 3 to 10 are represented by the same reference numerals and signs, and detailed descriptions thereof may not be repeated.

Referring to FIG. 15, a gas box 10A according to some embodiments of the present disclosure may include the gas line 11, the purge supply 12, a first gas valve 13, the flow controller 14, a second gas valve 15, and a third gas valve 16.

Each of the gas valves 13, 15, and 16 may include a plurality of gas valves. In example embodiments, the first gas valve 13 may be referred to as an upstream valve located upstream of the flow controller 14, the second gas valve 15 may be referred to as a downstream valve located downstream of the flow controller 14, and the third gas valve 16 may be referred to as a final gas valve located for example, at an end of the gas box, on the downstream side of the gas box 10A.

Referring to FIG. 16, Step S3030 of supplying gas from the gas box to the inspection device may include Step S3230 of the first gas valve repeatedly opening and closing the gas line. For example, Step S3030 may be executed instead of Step S3000 of the method S100 of FIG. 8.

The purge gas may be supplied in a state where all valves and flow controllers provided on the gas line are open. The purge gas may be provided to have an amount sufficient to saturate the gas line. A high pressure may be formed upstream of the gas line (for example, upstream of the first gas valve). The gas is discharged from the inspection device through the chamber or the foreline. Therefore, a relatively low pressure may be formed downstream of the gas line (for example, downstream of the first gas valve). For example, a pressure difference may be formed upstream of the first gas valve and the downstream side of the first gas valve. In such a manner, a relatively large pressure difference may occur between the upstream side and the downstream side of the first gas valve.

In a case where the first gas valve is opened and closed, the purge gas may pass through the first gas valve at a high speed due to the pressure difference. For example, the first gas valve may be repeatedly opened and closed 10 times for about 100 seconds, or opened and closed 8-12 times for 80-120 seconds. By repeating the opening and closing in such a manner, contaminated particles or residual gas on the inside of the first gas valve and the wall surface of the gas pipe may be cleaned. The purge gas may carry the particles or residual gas to the inspection device.

Step S3030 of supplying gas from the gas box to the inspection device may include Step S3330 of repeatedly opening and closing the gas line through the flow controller.

A high pressure may be formed upstream of the gas line (for example, upstream of the flow controller). Gas is discharged from the inspection device through the chamber or the foreline. Therefore, a relatively low pressure may be formed downstream of the gas line (for example, downstream of the flow controller). A relatively large pressure difference may occur between the upstream side and the downstream side of the flow controller.

In a case where the flow controller is opened and closed, the flow rate is adjusted to 100% and 0%, the purge gas may pass through the flow controller at a high speed due to the pressure difference. For example, the flow controller may be repeatedly opened and closed 10 times for about 200 seconds, or opened and closed 8-12 times for 180-220 seconds. By repeating the opening and closing in such a manner, contaminated particles or residual gas on the inside of the flow controller and the wall surface of the gas pipe may be cleaned. The purge gas may carry the particles or residual gas to the inspection device.

Step S3030 of supplying gas to the inspection device may include Step S3430 of repeatedly opening and closing the gas line through the second gas valve.

A high pressure may be formed upstream of the gas line (for example, upstream of the second gas valve). Gas is discharged from the inspection device through the chamber or the foreline. Therefore, a relatively low pressure may be formed downstream of the gas line (for example, downstream of the second gas valve). A relatively large pressure difference may occur between the upstream side and the downstream side of the second gas valve. In a case where the second gas valve is opened and closed, the purge gas may pass through the gas valve at a high speed due to the pressure difference. For example, the second gas valve may be repeatedly opened and closed 10 times for about 100 seconds, or opened and closed 8-12 times for 80-120 seconds. By repeating the opening and closing, contaminated particles or residual gas on the inside of the second gas valve and the wall surface of the gas pipe may be cleaned. The purge gas may carry the particles or residual gas to the inspection device.

Step S3030 of supplying gas to the inspection device may include Step S3530 of repeatedly opening and closing the gas line through the third gas valve.

A high pressure may be formed upstream of the gas line (for example, upstream of the third gas valve). The gas is discharged from the inspection device through the chamber or the foreline. Therefore, a relatively low pressure may be formed downstream of the gas line (for example, downstream of the third gas valve). A relatively large pressure difference may occur between the upstream side and the downstream side of the third gas valve. In a case where the third gas valve is opened and closed, the purge gas may pass through the third gas valve at a high speed due to the pressure difference. For example, the third gas valve may be repeatedly opened and closed 10 times for about 100 seconds, or opened and closed 8-12 times for 80-120 seconds. By repeating the opening and closing in such a manner, contaminated particles or residual gas on the inside of the third gas valve and the wall surface of the gas pipe may be cleaned. The purge gas may carry the particles or residual gas to the inspection device.

In such a manner, by using the inspection methods according to some embodiments of the present disclosure, it is possible to detect which part of the gas box is contaminated.

Although the present disclosure has been described above by example embodiments and drawings, the present invention is not limited thereto. Thus, it is apparent that various modifications and variations may be made by those skilled in the art relating to the present disclosure without departing from the scope of the technical idea of the present disclosure and the equivalent scope of the patent claims to be described later.

What is claimed is:

1. An inspection system comprising:
a gas box configured to store gas;
an inspection device configured to receive gas supplied from the gas box to the inspection device and configured to inspect the gas received from the gas box, and
an inspection line connecting the gas box to the inspection device and configured to allow gas supplied from the gas box to flow through the inspection device;
wherein the inspection device comprises:
a first sensor configured to detect particles present in the gas flowing through the inspection line; and
a second sensor configured to detect target gas present in the gas flowing through the inspection line.

2. The inspection system according to claim 1, wherein the inspection line comprises:
an inlet port connected to the gas box and configured to introduce the gas from the gas box to the inspection device;
an inlet valve configured to selectively open and close the inlet port;
a first outlet port disposed downstream of the first sensor and the second sensor and configured to discharge the gas introduced to the inspection device through the inlet port; and
an outlet valve configured to selectively open and close the first outlet port.

3. The inspection system according to claim 2, wherein the inspection line further comprises:
a first first sensor valve disposed upstream from the first sensor and a first second sensor valve disposed downstream from the first sensor; and
a second first sensor valve disposed upstream from the second sensor and a second second sensor valve disposed downstream from the second sensor.

4. The inspection system according to claim 2, wherein the inspection system further comprises:
a pressure gauge disposed downstream of the inlet valve.

5. The inspection system according to claim 2,
wherein the inspection system further comprises:
a chamber that is supplied with the gas from the gas box to process a substrate, and
wherein the first outlet port of the inspection line is connected to the chamber to discharge the gas introduced to the inspection device through the inlet port of the inspection line, into the chamber.

6. The inspection system according to claim 5, further comprising:
a foreline connected to the chamber and configured to discharge gas from the chamber,
wherein a second outlet port is connected to the foreline to discharge the gas into the foreline.

7. The inspection system according to claim 1,
wherein the gas box comprises:
a gas line configured to allow the gas to move therethrough;
a gas valve configured to selectively open and close the gas line;
a flow controller configured to control a flow rate of the gas moving through the gas line; and
a purge supply configured to supply a purge gas to the gas line to purge the gas line, and
wherein the purge supply is disposed upstream of the gas valve and the flow controller.

8. The inspection system according to claim 7, further comprising:
a controller configured to control the gas box and the inspection device,
wherein the controller is further configured to:
control at least one selected from the group consisting of the gas valve, the flow controller, and the purge supply to control a flow rate of the gas flowing in the gas line, and
determine whether the gas box is contaminated based on an output data which is output from at least one of the first sensor or the second sensor.

9. The inspection system according to claim 8, wherein the controller is configured to:
control the gas valve such that the gas valve closes the gas line after the purge supply purges the gas line with purge gas, and
controls the gas valve such that the gas valve opens the gas line to introduce the purge gas into the inspection device through the gas line.

10. The inspection system according to claim 8, wherein the controller is configured to:
control the flow controller such that the flow controller closes the gas line after the purge supply purges the gas line with purge gas, and
control the flow controller such that the flow controller opens the gas line to introduce the purge gas into the inspection device through the gas line.

11. A substrate processing apparatus comprising:
a chamber for processing a substrate;
a gas box configured to store gas to be supplied to the chamber;
a foreline connected to the chamber configured to discharge the gas from the chamber;
an inspection device configured to connect at least one of the foreline or the chamber to the gas box, and
an inspection line connecting the gas box to the inspection device and configured to allow the gas supplied from the gas box to flow through the inspection device;
wherein the gas box comprises:
a gas line configured to allow the gas to move therethrough;
a gas valve configured to selectively open and close the gas line;
a flow controller configured to control a flow rate of the gas moving through the gas line and disposed downstream of the gas valve; and
a purge supply configured to supply a purge gas to the gas line to purge the gas line, and disposed upstream of the gas valve and the flow controller, and
wherein the inspection device comprises:
a particle sensor configured to detect particles present in the gas flowing through the inspection line; and
a gas sensor configured to detect target gas present in the gas flowing through the inspection line.

12. An inspection method comprising:
connecting an inspection device with a gas box;
connecting the inspection device with at least one of a chamber or a foreline connected to the chamber;
supplying gas from the gas box to the inspection device; and
determining whether the gas box is contaminated, through the inspection device.

13. The inspection method according to claim 12, wherein the gas box comprises:
a gas line configured to allow the gas to move therethrough;
a first gas valve configured to selectively open and close the gas line;
a flow controller configured to control a flow rate of the gas moving through the gas line; and
a purge supply configured to supply purge gas to the gas line to purge the gas line and disposed upstream of the first gas valve and the flow controller.

14. The inspection method according to claim 13, wherein the supplying of the gas from the gas box to the inspection device comprises:
supplying the purge gas to the gas line by the purge supply;
closing the gas line by the first gas valve; and
opening the gas line by the first gas valve.

15. The inspection method according to claim 13, wherein the supplying of the gas from the gas box to the inspection device comprises:
supplying the purge gas to the gas line through the purge supply;
closing the gas line by the flow controller; and
opening the gas line by the flow controller.

16. The inspection method according to claim 13, wherein the first gas valve is disposed upstream of the flow controller; and
further comprising a second gas valve disposed downstream of the flow controller.

17. The inspection method according to claim 16, wherein the supplying of the gas from the gas box to the inspection device comprises:
supplying the purge gas to the gas line by the purge supply;
closing the gas line by the first gas valve, the second gas valve, and the flow controller;
opening and closing the gas line repeatedly by the first gas valve;
opening and closing the gas line repeatedly by the flow controller; and
opening and closing the gas line repeatedly by the second gas valve.

18. The inspection method according to claim 12, wherein the inspection device comprises:
a particle sensor configured to detect particles present in the gas flowing through an inspection line connecting the gas box to the inspection device and configured to allow gas supplied from the gas box to flow through the inspection device; and
a gas sensor configured to detect target gas present in the gas flowing through the inspection line.

19. The inspection method according to claim 18, wherein the determining of whether the gas box is contaminated in the inspection device comprises:
determining that the gas box is contaminated where either the particle sensor detects particles in the gas flowing through the inspection line and/or the gas sensor detects the target gas in the gas flowing through the inspection line.

20. The inspection method according to claim 18,
wherein the inspection device further comprises
a pressure gauge disposed in the inspection line and configured to measure a pressure of the gas flowing through the inspection line, and
wherein the inspection method further comprises:
checking whether outside air is introduced to the inspection line based on a measured value of the pressure gauge.
